(19) United States
(12) Patent Application Publication
(10) Pub. No.: US 2004/0142371 A1
(43) Pub. Date: Jul. 22, 2004

(54) Process for requesting biological experiments and for the delivery of experimental information

(75) Inventors: Aleksandar Milosavljevic, The Woodlands, TX (US); Mitchell Donn Eggers, The Woodlands, TX (US); Michael E. Hogan, Conroe, TX (US); Deval Arun Lashkari, The Woodlands, TX (US); Robert H. Ellis, Kemah, TX (US)

Correspondence Address: Robert D. Touslee, GMX Technology, Inc., 29 Golden Eagle Lane, Littleton, CO, 80127, US

(73) Assignee: GENOMETRIX GENOMICS INC.

(21) Appl. No.: 10/751,069

(22) Filed: Jan. 2, 2004

Related U.S. Application Data
(63) Continuation of application No. 10/613,434, filed on Jul. 3, 2003.
Application No. 10/613,434 is a continuation of application No. 09/632,539, filed on Aug. 4, 2000, now abandoned.
(60) Provisional application No. 60/161,694, filed on Oct. 26, 1999.

(51) Int. Cl. C12Q 1/68; G06F 17/60; G06F 19/00; G01N 33/48; G01N 33/50

(52) U.S. Cl. 435/6; 702/20; 705/3

(57) ABSTRACT

A method is disclosed for requesting genomics services from a service provider over the Internet and providing genomics services to a client over the Internet. The client provides biological samples and identifies genome sequences of interest. The service provider obtains the biological samples and genome sequences, provides microarrays containing the identified genome sequences, and applies the biological samples to the microarrays. The client receives the results of the analysis over the Internet. The genomics services provided include genotyping, gene expression, and proteomics. The Client is able over the Internet to obtain the current status of the samples and the experiments and to modify the requested experiments based, for example, on the analysis of previously obtained results.

### RELATED APPLICATIONS

This application is based on U.S. Provisional Application 60/161,694, filed Oct. 26, 1999, and incorporated herein by reference.

### FIELD OF THE INVENTION

This invention relates to processes for requesting experiments involving macromolecular recognition events and for obtaining and analyzing the information obtained from such experiments. More particularly, this invention relates to a process for requesting genomics information over the Internet, tracking and possibly modifying that request, and obtaining the results over the Internet for analysis.

### Glossary of Biological Terms

As the following words and phrases relate to this invention, they are to be understood as follows:

By “experiment” is meant a biological or chemical experiment involving at least one specific macromolecular recognition event. An experiment is defined by a particular assay and at least one sample on which the assay is to be performed.

By “macromolecular recognition event” is meant chemical interaction (colvalent or noncovalent bond) of a macromolecule or a mixture comprising at least one macromolecule such as DNA, RNA, polypeptide, or protein on one side and another organic or inorganic molecule on the other side.

By “capture probe,” “probe molecule,” or “probe” is meant a specific molecule involved in at least one macromolecular recognition event or a mixture comprising at least one molecule that is involved in at least one macromolecular recognition event. Examples of probes include PCR primers, oligomer probes immobilized on array surfaces or to other probes, and flourescently labeled antibodies. Probes are meant to include nucleic acids, DNA, RNA, receptors, ligands, antibodies, anti-antibodies, antigens, proteins, and also small chemical compounds such as drugs, haptens, or peptides. The informational content of macromolecular probes may be defined by their sequences, e.g. a sequence of nucleic acids for DNA probes or a sequence of amino acids for peptide probes.

By “experimental protocol” is meant a description of steps taken in the course of performing a particular experiment. An experimental protocol may be very specific, describing the detailed quantities of reagents used and the detailed steps, or it may be defined genes for example, “perform PCR” or “perform DNA hybridization.” An experimental protocol does not include the specification of the informational content of probes involved in macromolecular recognition events. For example, while a particular protocol for performing PCR may specify the approximate lengths of DNA primers or the number of distinct primers, it does not specify primer sequences. Similarly, a protocol for DNA hybridization may specify the approximate length of oligomer probes used, but it does not specify their sequence.

By “assay” is meant a set of instructions for carrying out an experiment on any particular sample, including the experimental protocol and the specification of probes used in the context of the protocol. An assay does not include the specification of samples on which the experiment is to be performed.

By “target molecules or target analyte” is meant the molecules of interest in a substance that are to be interrogated by binding to the capture probes immobilized in an array.

By “genotyping” is meant the process of analyzing the genetic makeup or genetic constitution of the studied organism. For instance, genotyping is used to locate the presence of specific base changes within a given individual's DNA sequence. Genotyping may be used, for example, to gather information regarding an individual's predisposition to a particular health condition or to confirm a diagnosis of genetic disease.

The term “phenotype” refers to the observable properties of an organism resulting from an interaction between the organism's genotype and the environment.

By “PCR” is meant a technique for synthesizing large quantities of specific DNA segments consisting of a series of repetitive cycles, including denaturation of the double-stranded DNA molecule, annealing of oligonucleotide DNA primers, and DNA polymerization.

By “PCR primer” is meant an oligonucleotide used to bind a complementary single-stranded DNA molecule and prime synthesis of the DNA complementary strand by DNA polymerase.

The term “array” refers to a spatial grouping or an arrangement.

The term “probe array” refers to the array of N different biosites deposited on a reaction substrate that serve to interrogate mixtures of target molecules or multiple sites on a single target molecule administered to the surface of the array.

By “array fabrication” is meant the process of preparing a substrate surface and depositing the appropriate probe to that surface in a particular spatial grouping or arrangement through the use of many different mechanisms, e.g., inkjet deposition, capillary, and photolithography. The term “oligonucleotide probe arrays” refers to the probe arrays wherein the probes are constructed of nucleic acid.

By “throughput” is meant the number of analyses completed in a given unit of time.

By “SNP” is meant Single Nucleotide Polymorphism.

By “mRNA target molecule” or “mRNA target analyte” is meant a substance containing identical mRNA components or a mixture of disparate mRNAs.

By “gene expression” is meant a multi-step process, including regulation of that process, by which the product of a gene is synthesized. More particularly, the DNA sequence of a gene is used to transcribe a complementary RNA sequence, and the RNA sequence is then translated into protein, which is responsible for carrying out normal cellular function. The study of gene expression generally refers to the analysis of RNA levels within a particular biological sample. Specifically, RNA is an intermediary species in the process of gene expression. As such, the relative level of RNA generally provides a proper indicator of the level of gene expression. Studying the level of gene expression provides a valuable method to analyze the effects of various compounds upon a biological system.

By “gene expression study” is meant an analysis of the process by which the product of a gene is synthesized. Specifically, the study detects the presence of an RNA molecule synthesized from the gene of interest. The analysis can be achieved by one of the many different methods known to those skilled in the art.

By “genotyping study” is meant an analysis of the genetic makeup of an organism. Specifically, the study detects genetic variation. The analysis includes, without limitation, detection of Single Nucleotide Polymorphisms, DNA sequence deletions, DNA sequence translocations, DNA sequence rearrangements, and DNA sequence repeats.

By “pharmacogenetics” is meant the study of the biochemical and physiologic aspects of drug effects, including without limitation, absorption, distribution, metabolism, elimination, toxicity, and mechanisms of drug action in relation to the genetic makeup of the organism.

By “genetic epidemiology” is meant the study the relationship between genetics and disease in populations.

By “allele” is meant alternative forms of a gene. Thus, an allele can represent one of several possible mutational forms of a gene, which is positioned at the same location on a chromosome.

The term “allele scores” refers to data defining the particular alleles detected upon analysis. An allele score may represent, without limitation, the number of specific alleles of interest detected within a single organism, or the number of organisms within a given study that have the specific allele or alleles of interest.

The term “expression scores” refers to data defining the expression levels of a gene, or set of genes.

The term “dot-scoring” refers to the measurement of a signal that comes from the interaction of a probe with target molecules.

The term “bio-scoring” refers to the biologically meaningful quantities and any other biologically meaningful information extracted from dot scores. Examples of bioscores include expression scores and allele scores.

The term “hybridization” is meant to include, without limitation, a means of two or more components to interact through hybridization, an association, linking, coupling, chemical bonding, covalent association, lock and key association, or reaction. For the purposes of this invention, these terms are used interchangeably.

By “material kitting” is meant the process of putting together the necessary materials required to conduct a particular step in a study, including without limitation, array fabrication and CR/Labeling.

By “bar code” is meant any recognizable identifier that corresponds to information that may be digitally stored and referenced. For example, one example of a bar code is the representation of characters by sets of parallel bars of varying thickness and separation that may be read optically by transverse scanning.

### BACKGROUND

Limitations of Traditional Biological Research

Traditional biological research is plagued by numerous technical as well as logistical problems. For example, genotyping and gene expression requires a large capital investment as well as a high level of labor and technical expertise. Another problem involves a researcher's inability to master relevant protocols in biological research. In particular, while a researcher's strength primarily centers about an ability to envision new relevant types of experiments, most of a researcher's productive time is typically spent mastering complex experimental protocols. Advanced techniques such as microarray fabrication for gene expression analysis, SNP detection, and proteomics typically require significant up-front costs, expensive reagents, and lengthy and costly training. These problems are exacerbated by an increasing rate of innovation that causes existing protocols to become obsolete at an ever increasing pace.

A second problem is the researcher's inability to procure the specific number of test samples necessary to perform a statistically meaningful population study. In the field of human study, this particular problem is exacerbated by ethical and privacy concerns and related institutional protocols, which generally restrict researchers to a limited number of unadulterated human samples. As compared to academic research, commercial researchers may incur even greater restrictions upon their ability to test a statistically significant number of unadulterated samples. As a result of problems associated with sample collection, most biological research is performed using in vitro cell and tissue cultures. In vitro samples, however, are well known to include large numbers of genetic variations or mutations. Experimental data obtained with these samples are frequently not statistically relevant to the population as whole, or even a sub-population.

A third problem is a researcher's inability to analyze the large data sets produced by the researcher's experiments. Although a researcher is typically quite adept at placing experimental data in the context of existing knowledge, the amount of data at the researcher's disposal may be overwhelming. This problem is exacerbated by two recent trends: (1) high-throughput microarray experiments that provide vast information about DNA polymorphisms, gene expression, and proteins, and (2) the growth of publicly accessible data sets, including the exponential growth of DNA sequence, SNP polymorphism, and gene expression information.

It is therefore very desirable to provide a researcher performing basic and applied research a process for requesting that specific assays, including specific probes, be designed and made available to the researcher for carrying out experiments. In addition, it is desired that a researcher be provided tools to access, search, and request specific samples from a biological repository (i.e., DNA repository) and its associated repository database, including relevant medical documentation. Providing the researcher with fast and simple access to experimental services, a biological repository database, and analytical tools will lead to a rapid accumulation of genetic knowledge. This would greatly benefit the development of personalized therapeutics, the future of medicine. Such a process would enable researchers to study thousands of samples and analyze the functional relevance of individual genetic variations through linkage to clinical outcomes.

Genomics

Genomics refers to the study of genes and their function. Recent advances in genomics ate bringing about a revolution in our understanding of the molecular mechanisms of disease, including the complex interplay of genetic and environmental factors. Genomics is also stimulating the discovery of breakthrough healthcare products by revealing thousands of new biological targets for the development of drugs, and by giving scientists innovative ways to design new drugs, vaccines, and DNA diagnostics. Genomics based therapeutics include traditional small chemical drugs, protein drugs, and gene therapy.

Genetic information provides the basis for understanding biological and physiological functions in organisms. This information is encoded in deoxyribonucleic acid (DNA), which is the basis of the inherited characteristics in organisms. Each DNA molecule is comprised of a combination of four nucleotide bases, adenine, guanine, cytosine, and thymine, which are generally referred to by the letters A, G, C, and T, respectively. Each nucleotide base is arranged along one of two complementary strands in a DNA molecule, each A pairing with T and each G pairing with C, with each such pair being called a base pair. The order of the bases along a DNA strand is called the DNA sequence, and the entire DNA content of an organism is called its genome. Within the genome are defined DNA sequences that form particular units of heredity. These units are generally referred to as genes. Differences of one or more bases in a gene, referred to as genetic variation, can modify how a gene functions by altering the protein it encodes, which alters the protein's normal effect on cell function. Additionally, one or more base changes can occur outside of a gene yet still effect cell function by altering that gene's normal level of gene expression. Base changes that effect gene expression may be found in the gene itself, or in regulatory elements outside of the gene. More complicated genetic variations may involve base changes in a first gene, whereby that base change ultimately effects the expression of a critical second gene. Genetic variations are a primary component of all major diseases, including cancer, diabetes, and cardiovascular disease.

The most common types of genetic variation between individuals are Single Nucleotide Polymorphisms, which are referred to as SNPs. A SNP is a change in a single base pair along the DNA sequence, such as replacing a T with a G along one strand and its complementary A with a C along the other strand. It is estimated that there are 3 to 10 million SNPs inherited from each parent. Tens of thousands of SNPs must be measured in an individual to distinguish those SNPs that are medically relevant from those that are of no medical consequence. To better comprehend the genetic basis of disease and its treatment, the effect on biological function caused by individual genetic variation and the interplay between multiple genetic variations must be understood. In this regard, major efforts are currently underway to discover the genetic SNP variability that exists in the population and in turn to use this basic SNP data as a starting point to understand inherited personalized variation in disease risk and in therapeutic response.

The traditional drug discovery process is expensive, time consuming, risky, and labor intensive. Despite expenditures of billions of dollars, only a small fraction of the thousands of compounds screened by pharmaceutical companies are developed into commercial drugs. To develop new medical treatments and diagnostics based on genetic information, pharmaceutical companies require access to information establishing the relevant functional connections between genes, disease states, medical history, and pharmaceutical treatments.

There are six stages in the drug development process, and genomics plays an important role in each stage. The first stage is target identification, in which a determination is made whether a particular gene is a good target for further investigation. This stage is typically initiated with genomic studies involving DNA sequencing, genetic mapping, and RNA analysis, where RNA, or ribonucleic acid, is the nucleic acid that serves as an intermediary for translating DNA sequence information into proteins.

The second stage in the drug development process is target validation, in which an attempt is made to demonstrate whether an identified target has an effect on the course of a disease. Target validation uses a number of genomic techniques, including RNA analysis, protein analysis, and cell biology. It is important in this stage that candidate targets be investigated under many different experimental conditions to better understand and select optimal drug targets.

In the third stage, primary screening, drug leads are identified through large-scale testing of collections, or libraries, of compounds against validated targets. The goal of this stage is to find “hits,” i.e., to find those individual members of the compound library that bind to, inhibit, or activate a particular target. High throughput genotyping (the study of DNA variation) and gene expression (the analysis of gene activity) are needed to enable researchers to screen hundreds of genes.

The fourth stage is lead optimization, in which drug leads that are likely to have appropriate therapeutic properties are identified by conducting successive rounds of chemical alterations and biological tests. Similar to target validation, a number of techniques are used in the lead optimization stage, including protein analysis, cell biology, chemical synthesis, and other high throughput experiments. This stage may also involve the genotyping and gene expression of compounds for therapeutic activity in animal models of disease.

The fifth stage in the drug development process is preclinical development, in which compounds are tested in cell cultures and in animals to evaluate their safety, effectiveness, distribution throughout the body, and metabolization. Formulation tests determine the best delivery method as well as check for consistent quality in manufacturing. Expression analysis provides toxicity assessments by indicating the behavior of specific toxicity marker genes.

The sixth stage in the process is clinical development, in which the safety and efficacy of drug leads or pharmaceutical compounds are tested in humans. Because clinical trials are the most expensive stage of drug development, pharmaceutical companies need to be able to analyze the genetic makeup of each patient to determine or predict responsiveness or adverse reactions to particular drugs. Genotyping experiments may be used to identify each individual's likely response to a drug, thus maximizing the productivity of the trial and reducing overall costs.

Genomics is the key to reducing the risk, cost, and lead time of drug discovery and the development of personalized medicine. Pharmaceutical and biotechnology companies are actively incorporating genomics into each stage of the drug development process. Thus, there is a need for rapid, large scale, and systematic searches for panels of SNPs, coupled with lifestyle and clinical data for each person providing a sample. Such methods will facilitate the discovery of the predictive rules that relate individual patient data to disease risk and patient response to pharmaceutical therapy. The complexity of the data required, the process of risk factor discovery, and ultimately the ability to deliver these discoveries into clinical trials suggest a need for high throughput genetic and molecular analysis, high capacity bioinformatics using advanced statistical methodologies, automated high throughput sample handling, and large scale sample libraries.

Current methods for analyzing genomic data and studying SNPs are typically low throughput technologies that produce results with limited accuracy due to human error and small sample sizes. These procedures typically generate single data points per sample per experiment, such as the expression levels of a single gene or the identification of a single polymorphism. A significant amount of work is involved in preparing a sample, processing it, and generating the data for eventual analysis. The resulting data is not easily reproduced due to cost, time, and sample availability. Since the biological samples are often of limited quantity and number, researchers must be careful to use samples sparingly if more data may be needed for future experiments.

Current technologies are also limited by sample storage and tracking. Traditional sample storage methods involve storage of liquid materials or frozen tissue specimens and require costly, space-consuming, low temperature cryogenic facilities. Scalability of these storage methods is difficult and requires larger facilities and additional freezers. Further, sample tracking and retrieval are problematic since employing a fully robotic system under cryogenic conditions is costly and often impractical.

Thus, there is a need for more productive and more efficient genetic analysis methods that are not plagued by the problems associated with sample quality and quantity. To more rapidly enhance our genetic understanding, there is a need to provide research entities located in remote locations with rapid access to biological sample archives and sample testing services. Further, there is a need for more productive and efficient drug discovery methods and genomic information services that will allow pharmaceutical and biotechnology companies to develop significantly higher numbers of drug compounds each year.

Electronic Data Transfer

Certain embodiments of the present invention are directed to use over the Internet The Internet consists of a large number of computers connected via network links and communicating via standardized Internet protocols to form a global, distributed network. While the term “Internet” as used herein is intended to refer to what is commonly known today as the Internet, it is also intended to encompass private as well as public networks and any variations to those networks that may exist in the future, including changes and additions to existing standard protocols.

Computer users communicate and exchange information over the Internet using standardized protocols. The World Wide Web (“WWW”) provides a visual interface to facilitate this communication and exchange of information. The WWW allows a server computer, having set up a web site, to send text and graphical images in the form of web pages to a client computer for display or storage.

Web pages are typically written using Hyper-Text Markup Language (“HTML”). The software on the client computer that interprets and executes the commands contained in web pages is called a browser. In this context, the “client” denotes the computer using a browser to request and display information and the “server” denotes the computer responding to those requests by providing web pages. A single web page may contain data from a number of different servers. Examples of browsers include the Microsoft Internet Explorer and the Netscape Navigator. The client indicates to the browser a desire to view a particular web page by entering that web page's address, which is referred to as its Uniform Resource Locator (“URL”), into the browser. The browser then initiates a client computer request to a server asking that it transfer to the client the HTML file that defines the requested web page. When the requested web page is received by the client, the browser uses it to construct a visual image of the web page on the client's display monitor or to store a visual image of the web page on the client's computer. The web page contains various commands for displaying text, graphics, controls, background colors, and other display features. In addition, the web page may contain other URL addresses, called hot links, that point to other web pages at the server's web site or other web sites. In addition, web pages may contain form fields or other devices that permit the client to transmit data to the server computer and may contain audio or video objects as well. A web page may be larger than the image displayed on the client computer's monitor, in which case the browser supplies scroll bars that permit the client to view different portions of the web page.

Web page description languages other than HTML are either currently available or planned for future release. In addition, various extensions to the basic HTML standard have been developed to provide additional features. These extensions include WebBot components, Java applets, browser plug-ins, Dynamic HTML (“DHTML”), and ActiveX controls. These extensions, all well known in the art, permit web pages to offer capabilities and services far beyond that offered by HTML alone. Methods for using these and other extensions to design web pages are well known in the art.

A browser communicates with a web server over a transmission link that operates according to the Transmission Control Protocol/Internet Protocol (“TCP/IP”). For the majority of Internet communications, a browser communicating with a server over a TCP/IP link sends and receives information using the Hyper-Text Transfer Protocol (“HTTP”). Most web browsers also enable clients to access server resources and services using additional protocols such as File Transfer Protocol (“FTP”) and Telnet.

Communication between a client and server generally takes place over communication links such as telephone lines and public network lines that are not inherently secure. Two important security issues related to client-server communications are privacy and authentication. Privacy involves prohibiting anyone other than the intended recipient from being able to read a communication between the client and the server. Privacy is typically accomplished using cryptographic methods by which communications are encrypted prior to transmission and decrypted subsequent to receipt. One popular protocol for providing an encrypted communication link between the server and the client is the Secure Sockets Layer (“SSL”) protocol developed by Netscape Communications Corp. This protocol is typically referred to as the HTTPS protocol. Other security protocols are available, including Private Communications Technology (“PCT”), Secure Hyper-Text Transport Protocol (“SHTTP”), and Pretty Good Privacy (“PGP”). Methods for using these and other protocols to provide secure Internet connections are well known in the art.

A second important security issue related to client-server communications is authentication. Authentication involves verifying that the entity with whom a client (or server) is communicating is in fact the actual server (or client). One method of authentication uses certificates to authenticate a message. A certificate is a set of digital data that identifies an entity and verifies that the public encryption and signature keys included within the certificate belong to that entity. Methods of providing authentication are well known in the art.

Currently, the primary standard protocols for allowing applications to locate and acquire Web documents are HTTP and HTTPS, and the Web pages are encoded using HTML. However, the terms “Web” and “World Wide Web” as used herein are intended to encompass future markup languages and transport protocols that may be used in place of or in addition to HTML, HTTP, or HTTPS. Further, the, term “Web” as used herein is a broad term including, for example, the hardware and software server components as well as any non-standard or specialized components that interact with the server components to provide services to Web site users.

The detailed description of the present invention given below is presented largely in terms of procedures, steps, logic diagrams, and other symbolic and descriptive representations that depict the operations of data processing devices connected via networks. These process descriptions and representations are the methods used by those experienced or skilled in the art to most effectively convey the substance of their work to others skilled in the art. Certain steps require the physical manipulation of electrical signals that are capable of being stored, transferred, combined, compared, displayed, or otherwise manipulated in a computer system or network. For convenience and clarity these electrical signals may be referred to as bits, values, elements, symbols, operations, messages, terms, numbers, images, or the like. All of these and similar terms are merely convenient labels and are to be associated with the appropriate electrical signals to which they correspond. Unless specifically stated or otherwise apparent from the following description, the terms “processing,” “computing,” “displaying,” and the like refer to actions and processes of a computing system and network that manipulates and transforms data represented by electrical signals within the computing device's memory, where the term “memory” includes ROM, RAM, magnetic storage media, optical storage media, and the like.

### SUMMARY OF THE INVENTION

The present invention is directed toward the advancement of genomic studies by providing genomic services to academic and commercial researchers, drug manufacturers, and healthcare providers over the Internet. The present invention provides methods and systems for requesting experimental services from a service provider and providing experimental services to a client. The experimental request consists of an assay and a set of samples on which the assay is to be performed.

In one embodiment, the assay may include a multiplicity of probes. Some of the probes may be immobilized or otherwise identifiable while others, such as PCR primers, may be used in solution. In one embodiment, some of the probes used in the assay may be immobilized on a flat surface, such as glass. In another embodiment, some of the probes may be immobilized in a three-dimensional polymer. In yet another embodiment, some of the probes may be immobilized on bead surfaces. In one embodiment, the assay may be specified exactly by the client, either by the client providing a specific set of probes to be used in the assay or by the client selecting a pre-specified assay. In another embodiment, a list of pre-specified assays may be stored in the database and made accessible to clients through relational or keyword based queries or through a hierarchically organized catalog.

In another embodiment, instead of providing a specific assay, the client requests that an assay, including a set of particular probes, be designed based on a client-specified target of the experiment. One type of experimental target is a list of genes the client may be interested in examining for changes in expression levels, in which case a particular assay, including a set of oligonucleotide hybridization probes for use on microarrays, is designed prior to conducting the experiment. Another type of experimental target is a list of SNP polymorphisms the client may be interested in examining for variations across a patient population, in which case a particular assay, including a set of oligonucleotide hybridization probes, and a set of PCR primers for amplifying the number of polynucleotides containing polymorphic sites is designed prior to conducting the experiment. In yet another embodiment, the experimental target is a list of proteins, in which case an assay such as a multiplex ELISA assay, including a set of antibodies, is designed prior to conducting the experiment.

The client may provide one or more biological samples. The service provider then obtains the biological samples and genome sequences and carries out an assay by applying at least one of the biological samples to at least one of the microarrays. The client receives data representative of the results of the application of the biological samples to the microarrays. The biological samples may include tissue samples or blood samples. In another embodiment, the service provider provides a repository of biological samples and provides a catalog of that repository. The client then accesses the catalog and selects biological samples from the catalog.

Communication between the client and the service provider may occur over an Internet connection, which may be a secure connection. In a further embodiment, the data representative of the application of the biological samples to the microarrays may first be analyzed by the client. In another embodiment the analysis is first performed by the service provider.

The step of providing biological samples may include transporting the samples from the client to the service provider or selecting biological samples from a repository under the control of the service provider. The data may include gene expression data, and the biological samples may include total RNA samples or poly-A RNA samples. In an alternate embodiment, the client provides one or more biological samples. The service provider obtains the samples and applies at least one of the biological samples to the microarrays. The client receives data representative of the applying step.

The present invention overcomes the limitations of current methods of providing genomic services by providing a highly integrated on-line technology platform that combines in a preferred embodiment assay services, a biological repository database with high throughput microarray analysis, automated sample handling, high capacity bioinformatics, and statistical genetics. The disclosed platform provides on-line cost effective genotyping, gene expression analysis, and proteomics, thereby assisting pharmaceutical and biotechnology companies in their efforts to develop safer, more effective therapeutics. While a preferred use of the disclosed platform is to develop more effective therapeutics, the scope of the present invention is not to be so limited. Specifically, the present invention can be used by any client desiring to obtain experimental services.

By using the present invention, a client has the ability to design an experiment from a remote location, submit that experiment for processing, and then obtain the results of that experiment delivered to the remote location. Communication to and from the remote location may occur over a standard Internet connection. The experiment is conducted according to an integrated process that may include integrated purchasing processes for acquiring the necessary materials from outside vendors as well as laboratory sample and status tracking routines. The sample and array tracking may be linked intelligently to material requisition software and lab information management software. The samples and arrays are identified for tracking, preferably digitally indexed, enabling a client to remotely query the status of the subject assay. Further, the present invention includes capabilities permitting a client to remotely modify and redesign previously submitted experiments based, for example, on the results of other or past experiments.

Once the experiments are performed, the researcher may be provided tools to access the experimental data and analyze it in the context of biological information accessible through the computer. Once the analysis stage is completed, the researcher may be enabled to design and request new experiments that are based on the results of the analysis of previously completed experiments.

### BRIEF DESCRIPTION OF THE DRAWINGS

FIG. 1 illustrates a connection between a Client and a Service Provider over the Internet.

FIG. 2 illustrates an embodiment of the present invention directed toward providing genotyping services.

FIG. 3 illustrates an embodiment of the present invention directed toward providing gene expression services.

FIG. 4 illustrates an embodiment of the present invention directed toward providing proteomic services.

FIG. 5 illustrates a microarray suitable for use with the present invention.

FIG. 6 illustrates a user interface for analysis software suitable for use with the present invention.

FIG. 7 illustrates a division of tasks between a Client and Service Provider in one embodiment of the present invention.

FIG. 8 illustrates a data entry screen for entering a genotyping project according to one embodiment of the present invention.

FIG. 9 illustrates a data viewing screen according to one embodiment of the present invention.

FIG. 10 illustrates the flow of data according to one embodiment of the present invention.

FIG. 11 illustrates the steps involved in a typical genotyping experiment according to one embodiment of the resent invention.

FIG. 12 illustrates a Production Menu according to one embodiment of the present invention.

FIG. 13 illustrates a Source Plate Importation Screen according to one embodiment of the present invention.

FIG. 14 illustrates a Work Order Window according to one embodiment of the present invention.

FIG. 15 illustrates a Plate Detail Window according to one embodiment of the present invention.

FIG. 16 illustrates a Sample Detail Window according to one embodiment of the present invention.

FIG. 17 illustrates a BioScore Computation Window according to one embodiment of the present invention.

FIG. 18 illustrates a Process Flow Chart in accordance with an embodiment of the present invention.

### DETAILED DESCRIPTION

### Description of Experimental Services

The present invention can be divided into five aspects. (1) Sample Acquisition; (2) Sequence or SNP Identification; (3) Array Fabrication; (4) Data Acquisition; (5) Data Delivery and Analysis. These aspects will be explained via their use with respect to four particular embodiments of the present invention: (1) Genotyping based on Client supplied samples; (2) Genotyping based on samples selected from a DNA repository database; (3) Gene Expression based on Client supplied samples; and, (4) Proteomics.

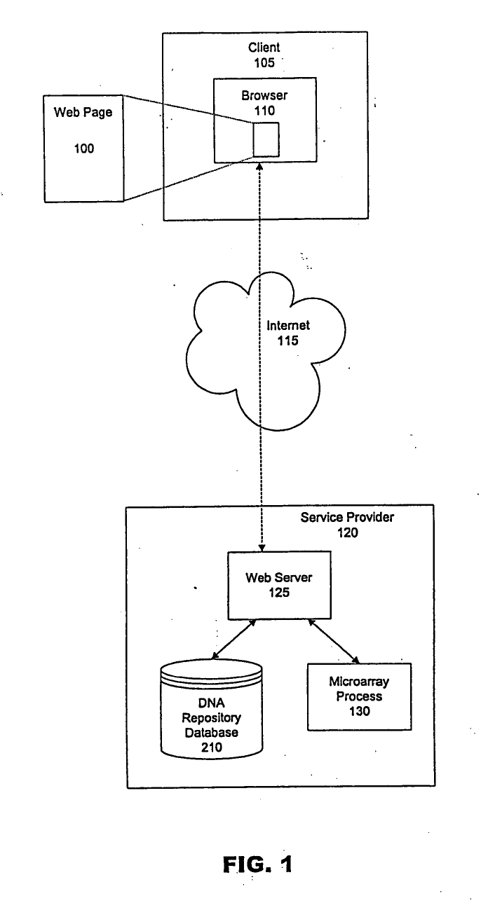

Referring to FIG. 1, Client 105 uses Browser 10 to request Web Pages 100 from Service Provider 120. This request is made over Internet 115 and is processed by Web Server 125. Web Server 125 may be connected to DNA Repository Database 210. Web Server 125 is further connected to Microarray Process 130 and configured to obtain results from that process for transmission to Client 105 as Web Pages 100. The basic framework of FIG. 1 is suitable for use with each of the following four embodiments of the present invention.

Genotyping: Client Supplied Samples

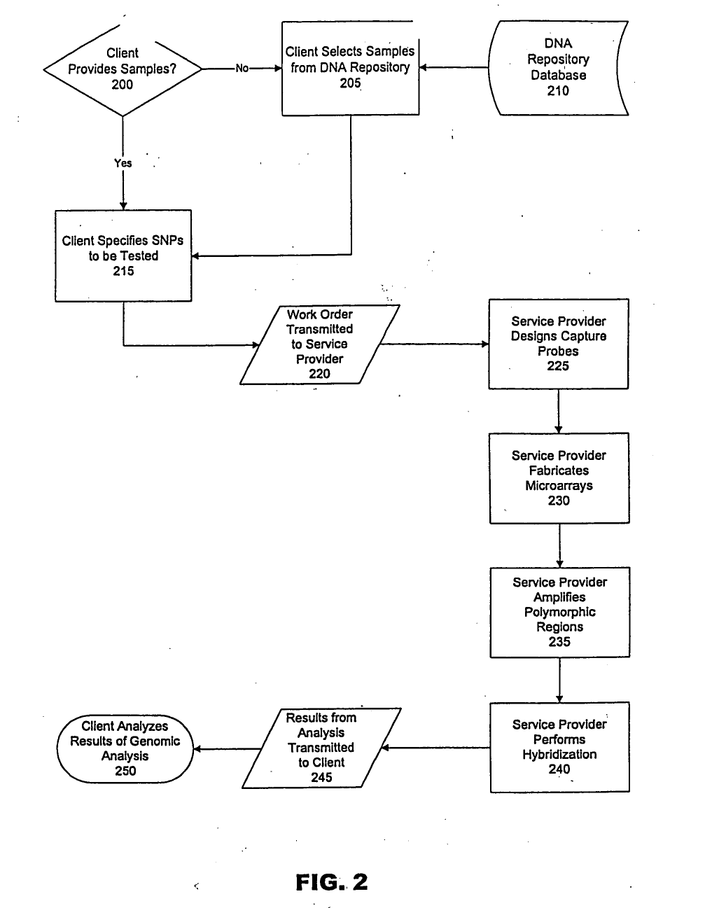

Referring now to the flow chart of FIG. 2, one embodiment of the present invention occurs when the Client provides its own samples in response to the query in Step 200 of FIG. 2. The Client next chooses the sequences or SNPs of interest in Step 215. (No specific ordering of the steps is required unless specifically stated. For example, Step 215 could occur before Step 200.) The sequences or SNPs that the Client desires to be analyzed may be submitted to the Service Provider via an on-line information system or via some other means such as an electronic spreadsheet or hard copy. The samples and the identified SNPs are submitted in Step 220 to the Service Provider as a work order. All interaction between the Client and the Service Provider may be transacted over the Internet, preferably using a secure encrypted protocol such as the HTTPS protocol described above. The submitted samples are preferably identified by a unique identification label or barcode.

After samples have been acquired and the sequences or SNPs of interest have been identified, the Service Provider provides suitable capture probes in Step 225. In Step 230, the Service Provider designs, fabricates, and validates custom microarrays and PCR primers. The PCR process amplifies the DNA in Step 235.

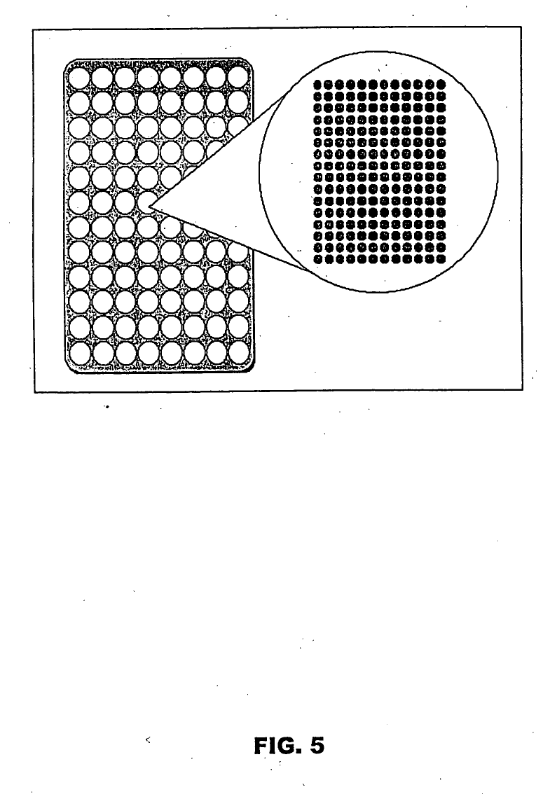

In Step 240, the Service Provider investigates or interrogates the samples using the prepared microarrays. The microarray may be fabricated using the capillary microarray manufacturing system disclosed in U.S. Pat. No. 6,083,763, which disclosure is incorporated herein by reference. Such system is capable of printing a microarray at high throughput. Other microarray types or hybridization or expression detection systems may be used, as the present invention is not limited to the use of any particular platform. A microarray consists of an array of test sites formed on a suitable structure. One example of a microarray is shown in FIG. 5. DNA or RNA capture probes of known binding characteristics are attached to each of the test sites. The probes in each test site differ from the probes in other test sites in a known manner. A sample containing an unknown candidate molecule, such as a DNA molecule containing a SNP, is hybridized to the microarray containing the capture probe. A signal is applied to the microarray so that certain electrical, mechanical, or optical projections of the test sites can be detected. A positive signal indicates that a molecular structure present in the sample substance has bound to a certain probe in one or more of the test sites of the array. The microarrays may be imaged using the microarray imager also disclosed in U.S. Pat. No. 6,083,763, which can image an entire plate of 96 microarrays within 60 seconds. Further, this system may be automated to minimize human error and permit rapid analysis. Customers may choose to use either standard or custom microarrays. Standard arrays are pre-fabricated and contain SNPs of general interest, such as, for example, those related to drug metabolism or cancer predisposition.

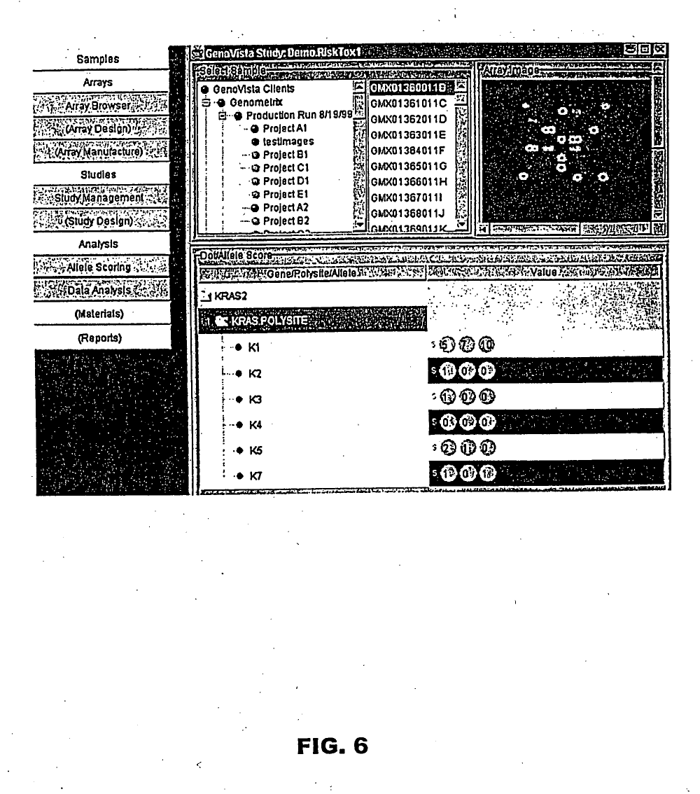

After the data is obtained, it is delivered in Step 245 to the Client for analysis in Step 250. The delivery in Step 245 preferably occurs using a secure on-line connection between the Client and Service Provider, but it may also occur by alternate methods. In addition, the analysis of the data in Step 250 preferably occurs subsequent to delivery to the Client, but it may occur prior to delivery as well. The analysis software may be resident in the Customer's computer or the Service Provider may provide on-line analysis that is controlled remotely by the Client. If the analysis is provided by the Service Provider, then the Client may receive none or only a portion of the actual data on which the analysis is based. If the data is analyzed by the Client, the analysis may be performed using data analysis software provided by the Service Provider or by standard commercial packages such as electronic spreadsheet software. The analysis software preferably provides large data set analysis and visualization functions. A sample user interface for analysis software suitable for use with the present invention is shown in FIG. 6.

It should also be understood, however, that the presently disclosed on-line technology platform is not limited to genotyping services that test only for SNPs. More particularly, a Client may request that genetic variations other than or in addition to SNPs be tested. For instance, an on-line Client can specify that the submitted samples, such as cancer specimens, be tested for multiple base DNA deletions and insertions using microarray analysis. Alternatively, a Client can request that submitted samples be analyzed for SNPs, deletions, insertions, and/or rearrangements. Alternate forms of genetic variation and methods of detecting them are well known in the art, and the present invention is not limited to any particular genetic or biological test.

Genotyping: Samples Selected From DNA Repository Database

In a second embodiment of the present invention shown in FIG. 2, the Client does not provide its own samples in Step 200, but instead selects samples from DNA Repository Database 210 in FIG. 2. Though the present embodiment discloses selection of DNA samples from a DNA Repository via access to a DNA Repository Database, the present invention is not so limited. For example, biological samples from a repository containing a wide-range of biological samples could be used. Such samples could include, without limitation, tissue, blood, skin, and hair.

The DNA Repository services are preferably provided directly by the Service Provider, but they may be provided directly by a third party or indirectly by the Service Provider as an offsite Sample Archive Service. DNA Repository Database 210 preferably includes biological sample information as well as an index to attendant clinical records associated with those samples. These records preferably are bar-coded and devoid of information that could lead to patient identification.

In one embodiment, the DNA samples in the DNA Repository are stored on cards according to the method described in Provisional U.S. Patent Application 60/161,694 filed on Oct. 26, 1999, which application is being incorporated by reference. When a particular sample is requested, automated equipment such as robotics may be used to select and obtain the corresponding DNA sample and provide the sample to the microarray for interrogation. However, other methods for DNA sample storage and access may be used in accord with the present invention.

The Client preferably uses a secure Internet connection to search through the sample records in DNA Repository Database 210. The Service Provider preferably provides access to the database via secure Web Pages, as described above. More specifically, the preferred embodiment permits the Client access to the database, even though that Client is remotely located relative to the DNA Repository itself.

In addition to a catalog of indexed biological samples, the repository database preferably includes corresponding clinical records, phenotypic information, and follow-on medical history. Following a search or analysis of this and other repository information, the Client selects a subset of samples for investigation. Once the Client selects the samples, the Client then designs the genotyping study on-line by selecting the SNPs of interest in Step 215. The analysis request is provided to the Service Provider as a work order in Step 220. The remaining steps in FIG. 2 proceed as in the previous embodiment.

In an alternate embodiment of the present invention, the Service Provider performs genotyping analysis of the samples in the DNA Repository prior to a request for that analysis by the Client. A Client may later access the results of that analysis, preferably using a secure Internet connection.

Gene Expression: Client Supplied Samples

In yet another embodiment, gene expression services maybe requested and provided over a secure Internet connection between the Client and Service Provider in a manner similar to that previously described for genotyping services.

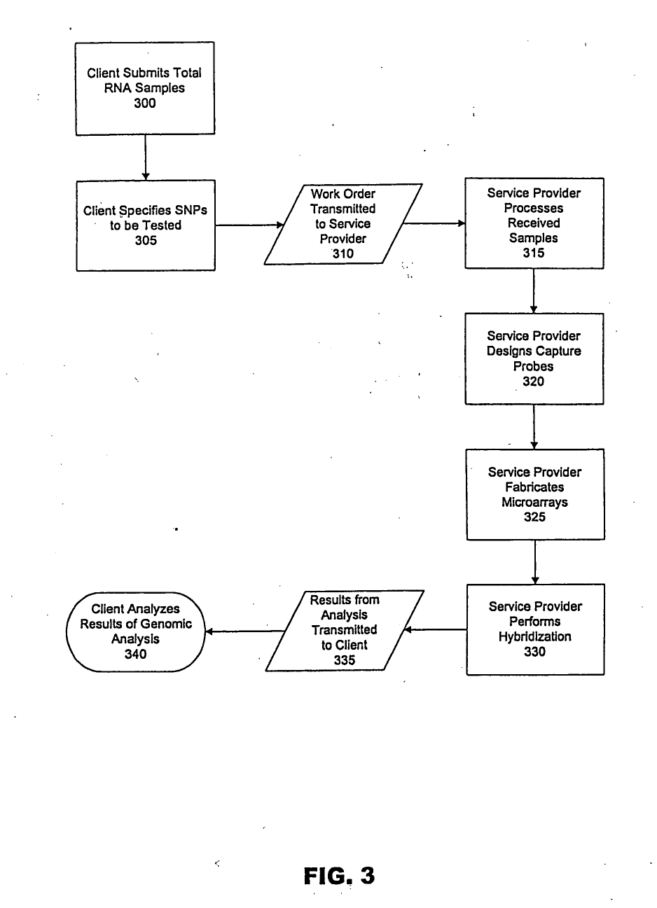

Referring to FIG. 3, the Client submits samples to the Service Provider in Step 300. The samples preferably comprise tissue culture cells, total RNA samples, and/or poly-A RNA samples. The Client identifies the SNPs of interest in Step 305 and submits that information to the Service Provider along with the samples as a work order in Step 310. The submitted samples are preferably identified by a unique identification label or barcode.

The Service Provider processes the received samples in Step 315 and generates the appropriate capture probes in Step 320. The Service Provider fabricates suitable microarrays in Step 325. In another embodiment, the Client chooses the microarrays from a collection of standard prefabricated microarray designs made available by the Service Provider. The Service Provider then performs the hybridization in Step 330 to interrogate the samples.

After the data is obtained, it is delivered in Step 335 to the Client for analysis in Step 340. The delivery in Step 335 preferably occurs using a secure on-line connection between the Client and Service Provider, but it may also occur by alternate methods. In addition, the analysis of the data in Step 340 preferably occurs subsequent to delivery to the Client, but it may occur prior to delivery as well. The analysis software may be resident in the Customer's computer or the Service Provider may provide on-line analysis that is controlled remotely by the Client. If the analysis is provided by the Service Provider, then the Client may receive none or only a portion of the actual data on which the analysis is based. If the data is analyzed by the Client, the analysis may be performed using data analysis software provided by the Service Provider or by standard commercial packages such as electronic spreadsheet software. The analysis software preferably provides large data set analysis and visualization functions.

Proteomics

The study of proteins and their function is called proteomics. In yet another embodiment, proteomic services can be requested and provided over a secure Internet connection between the Client and Service Provider in a manner similar to that previously described for gene expression and genotyping services.

A protein is a biological molecule consisting of a high molecular weight polypeptide chain of L-amino acids that is synthesized by living cells. Proteins are required for the structure, function, and regulation of cells, tissues, and organs in the body. The amino acid sequence of the polypeptide chain determines the proper structure and function of the protein. Any variation in the DNA gene sequence can cause the wrong amino acid to be synthesized into the polypeptide chain. An amino acid change can cause disruption of protein structure and function, and lead to a disease state. Additionally, aberrant gene expression can alter normal protein function. For instance, the level of protein within a cell can increase when the DNA gene sequence is over-expressed. This over-expression may be caused by a number of different factors, including drug response and gene mutation. Increases in protein levels can cause severe disruption of the increased protein, as well as of the function of other proteins. As a result, the over-expressed protein can cause a disease state or further exacerbate a disease condition.

A preferred method for detecting a specific protein (“antigen”) is to use an enzyme-linked immunosorbent assay (“ELISA”), which is well known in the art. An ELISA is useful for determining the presence or amount of antigen in samples ranging from crude cellular lysates to highly purified protein antigen preparations. An ELISA is able to detect native proteins or proteins with altered conformations. It should be understood by one skilled in the art, however, that various other methods of detecting antigens in a biological sample can be used without deviating from the scope of the present invention. For instance, ELISA protocols may differ significantly depending upon the specific antigen detected. It should be further understood that the term “antigen” includes proteins, drug molecules, viral particles, antibodies, and the like.

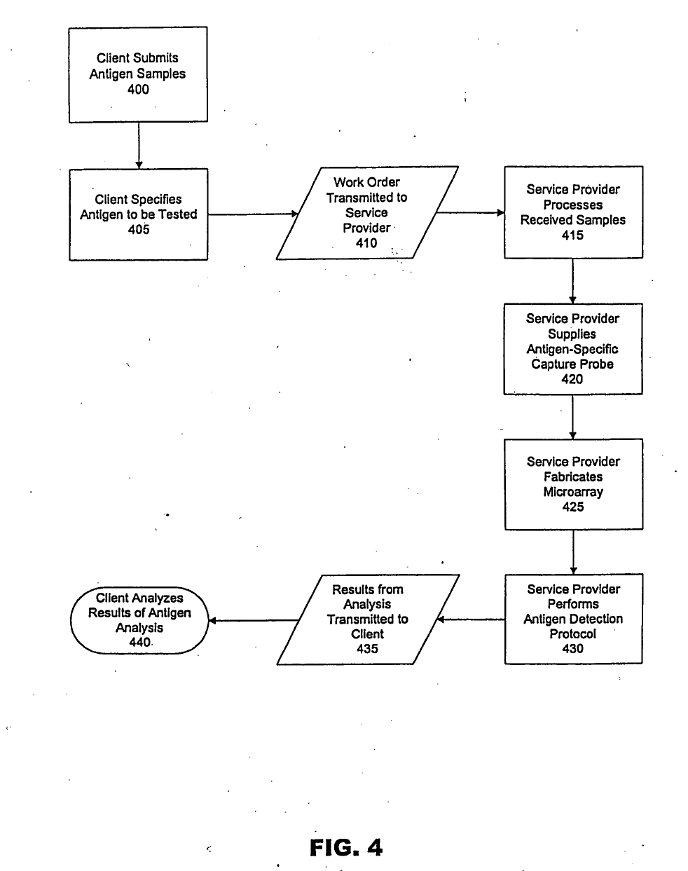

Referring to FIG. 4, the Client submits antigen samples to the Service Provider in Step 400. The samples preferably comprise solid tissue, tissue culture cells, crude or purified protein lysates, and the like. The Client specifies the antigen of interest in Step 405 and submits that information to the Service Provider along with the samples as a work order in Step 410.

If necessary, the Service Provider processes the received samples in Step 415. Methods to prepare antigen samples suitable for testing are well known in the art. For example, a purified antigen can be prepared using sodium dodecyl sulfate polyacrylamide gel electrophoresis (SDS-PAGE). SDS-PAGE provides a highly purified antigen sample suitable for sensitive detection and analysis protocols. Where native antigens are desired, a preferred electrophoretic purification method is a nondenaturing or “native” gel. Other suitable methods, however, are also well known in the art.

In Step 420, the Service Provider selects a capture probe operable to detect the antigen of interest designated by the Client in Step 405. It is well known in the art that the selected capture probe depends upon the antigen of interest. For instance, antibodies are generally used to detect a wide range of antigens, and vice versa. Antibodies are also used to detect other antibodies. Other combinations of probe based systems for detecting an antigen are well known in the biological arts. Using the selected capture probe, the Service Provider fabricates suitable microarrays in Step 425. One microarray for ELISA detection can be fabricated according to the description in “High-Throughput Microarray-Based. Enzyme-Linked Immunosorbent Assay (ELISA),” Mendoza et al., BioTechniques, 27:778-788 (October 1999), which is incorporated by reference herein. In another embodiment, the Client chooses the microarrays from a collection of standard prefabricated ELISA or other microarrays made available by the Service Provider. The Service Provider performs the antigen detection protocol (ie., ELISA) in Step 430 to interrogate the samples. The bound antigen may be detected according to the Mendoza article. However, other protein detection systems may be used to practice the present invention.

After the data is obtained, it is delivered in Step 435 to the Client for analysis in Step 440. The delivery in Step 435 preferably occurs using a secure on-line connection between the Client and Service Provide, but it may also occur by alternate methods. In addition, the analysis of the data in Step 440 preferably occurs subsequent to delivery to the Client, but it may occur prior to delivery as well. The analysis software may be resident in the Customer's computer or the Service Provider may provide on-line analysis that is controlled remotely by the Client. If the analysis is provided by the Service Provider, then the Client may receive none or only a portion of the actual data on which the analysis is based. If the data is analyzed by the Client, the analysis may be performed using data analysis software provided by the Service Provider or by standard commercial packages such as electronic spreadsheet software. The analysis software preferably provides large data set analysis and visualization functions.

### Description of System Software

This section describes an embodiment of System Software suitable for use with the present invention. The term “System Software” includes all of the software used by the Client to communicate with the Service Provider and obtain and analyze the data provided by the Service Provider including if applicable a browser suitable for the receipt of web pages and generic software such as an electronic spreadsheet program that may be used to analyze or visualize the received data. It also includes the software used by the Service Provider to provide genomics services to the Client and access to a repository database. The following description may be used by one of skill in the art to write System Software in accordance with the present invention. The software may be written using a variety of software platforms, but will typically include some HTML code and its various extensions for creating web pages supplied to the Client. Although the present invention is herein described in terms of various embodiments, it is not intended that the invention be limited to these embodiments. Modification within the spirit of the invention will be apparent to those skilled in the art.

Sample Submission

Software may be provided to permit secure, encrypted online access to genotype and gene expression information and analysis. The software may additionally provide access to operational processing activities and may facilitate the submission of samples and the analysis of results. The detailed description of the software system given below is presented largely in terms of procedures, steps, logic diagrams, and other symbolic and descriptive representations that depict the operations of data processing devices connected via networks. These process descriptions and representations are the methods used by those experienced or skilled in the art to most effectively convey the substance of their work to others skilled in the art.

Preferably samples are entered into the software system and assigned distinct barcodes prior to delivery to the Service Provider. In this way, the samples may be tracked throughout the various processes. The System Software will preferably be divided into integrated modules to aid high-throughput data collection and analysis. One module, for example, may be provided for SNP genotyping, pharmacogenics, and genetic epidemiology. Another module may be provided for gene expression at the mRNA level. Both modules preferably include analytical algorithms, querying capabilities, and visualization tools specifically designed for analyzing high-throughput genomics data. An export function may be provided to allow a Client to transfer data to its own database.

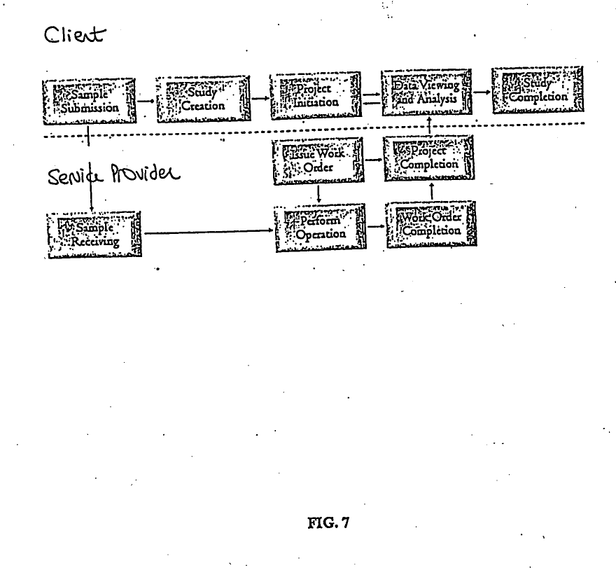

The software system facilitates communication between the Client and the Service Provider. The Client submits samples, creates studies, initiates projects, views raw data, analyzes data, and completes various studies. The Service Provider receives project requests from customers, issues work orders, logs completed work orders, and completes projects. Those receiving the work orders locate samples and perform operations on the samples. These roles are illustrated in FIG. 7. Although this embodiment has described various steps carried out by the Client and by the Service Provider, it would be possible for certain of the steps assigned to the Client to be carried out instead by the Service Provider and vice versa. In addition, certain steps could be carried out by a third party.

The following description applies to one embodiment of the system software that could be used in accordance with the present invention. This description is presented by way of example and is not intended to preclude other embodiments that could occur to one of skill in the art. Not all of the following steps will be required in every application.

The first step in the present embodiment of the system software is to obtain login information. The Client enters a User Name and a Password to obtain access to the system. Once access is granted, the Client is presented with an interface environment as, for example, is provided by the Microsoft Windows operating system. Access to the system may be granted on a number of levels depending upon the User Name. The contents of top-level and expanded menus may vary depending on the functional access permissions granted to a particular user. The level of permission may, for example, be based on the user's role in the process. A Client, for example, may be permitted access to the Sample Submission menu, the Study Management menu, the Data Viewing menu, and the Data Analysis menu. Various employees of the Service Provider may be permitted access to the Sample Receiving menu, the Sample Processing menu, and the Barcode Management menu.

Sample are submitted in units called “submissions” and each submission is assigned a unique submission code. Each submission may have multiple samples, each of which may be assigned a unique barcode. Prior to shipment, electronic submission is made via the Software System. Unique codes are assigned to each submission and to each sample within it for ease of tracking. Preferably, the barcodes are printed and affixed to the samples by the Client, but they may instead be printed and affixed by the Service Provider upon receipt. Also, preferably, each sample shipment should include a unique submission code. Each sample contained in the shipment should be uniquely identified either by a distinct client-issued identifier or, preferably, by an affixed barcode provided by the Service Provider. Upon receipt by the Service Provider, each shipment is identified by the enclosed submission code. The samples in the shipment may then be matched to the samples listed in the electronic submission to verify that all samples were successfully received.

The electronic submission of the sample via the System Software occurs prior to processing. This electronic submission preferably is done remotely by the Client prior to shipment, but may instead be done by the Service Provider upon receipt. Electronic sample submission preferably includes entry of the Client Name, the Work Station Operator Name, and the Sample Type. Data for each sample may be entered directly into the System Software or may be imported from an electronic spreadsheet such as Microsoft Excel or a database. The data entered for each sample preferably includes a Sample Number, a Patient/Organism ID, a Tube Identifier, and the Number of Units (i.e., the number of identical samples). A barcode may be generated by the System Software for each submitted sample. Once the data is entered it may be submitted over a network to the Service Provider. Further, the bar codes may be printed by the Client and applied to each sample.

The software preferably provides a tracking number by which the shipping status of the samples may be monitored. For example, a Federal Express tracking number may be assigned by the System Software to each sample submission. The status of the submission may then be viewed by opening the saved submission information and selecting the tracking number of interest. In this way the Client may determine if the samples are in transit or have been received by the Service Provider and entered into the sample database.

Upon receipt of the samples, the Service Provider accesses the System Software and enters a Receiving Operator Code as well as the Submission Code that was assigned when the samples were electronically submitted. Storage and substorage information may also be entered. If the Client did not apply barcodes, then barcodes may be generated and printed by the System Software and applied by the Receiving Operator at this time. Finally, the samples are placed in the assigned storage and substorage facilities and the sample database is updated.

Studies and Projects

The System Software may be designed to implement both studies and projects, where the term “study” represents a unit of scientific inquiry defined by the Client and the term “project” represents a unit of experimental data. A project is defined by a set of samples and a multiplexed biological assay that is performed on them within the context of a particular study. For example, three studies (Study 1, Study 2, and Study 3) could be based on three sample sets (Sample Set 1, Sample Set 2, Sample Set 3, respectively). Each study could be directed toward a subset of four assays (Assay 1, Assay 2, Assay 3, and Assay 4). Study 1 may include Project A directed toward Assay 1 and Project B directed toward Assay 2. Study 2 may include Projects C, D, and E directed toward Assays 2, 3, and 4, respectively. Finally, Study 3 may consist of Project F directed to Assay 3. Each project or study may be in any one of the following four stages, each of which is optionally associated with the indicated color code: (1) Created [blue], (2) Initiated [yellow], (3) In Process [orange], and (4) Completed [green].

Study management tools may be provided to allow the Client to create studies, initiate projects, and monitor the progress of the various projects and studies. In addition, Customers may be permitted to modify the projects and studies based on, for example, earlier obtained results. These tools may be initiated by a Study Management Window that lists all of the Customer's projects, either pending or completed. The Client may indicate on this screen those projects that are of interest.

To create a new study, the Client may choose the New Study option from the Study Management Menu. A window may then appear showing the Client name and permitting the Client to enter a name for the study, an objective for the study, and an experimental plan for the study. The new study will then appear in the Client/Study/Project Tree, which lists the projects associated with each study and the studies associated with the particular Client.

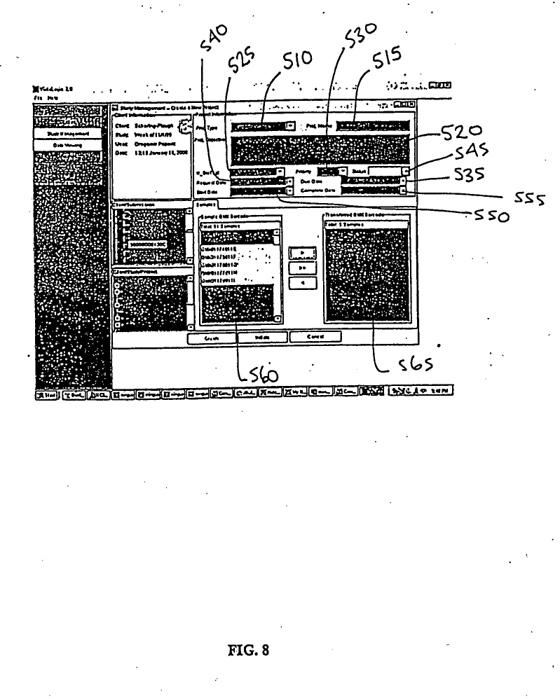

As an example, and referring to FIG. 8, a genotyping project may be entered according to the following procedure: (1) Select “Genotyping” as the Project Type in Field 510. (2) Enter the Project Name in Field 515. (3) Enter the Project Objective in Field 520. (4) Select the Bio Test in Field 525. (5) Select the Priority in Field 530. (6) Enter the Due Date in Field 535. The Request Date will be entered automatically into Field 540. As the project progresses, its status will be shown in Field 545 and the Start Date and Complete Date will be updated in Fields 550 and 555, respectively. The project may be created and saved without initialization or the project may be initialized during the creation process. To initiate the project, the Client accesses the Sample Submission menu to display a list of barcoded samples in Panel 560. The samples may then be chosen by barcode identifier, imported into the newly created project, and transferred to Panel 565. At this time the project may be initiated. Initialization of a Gene Expression project proceeds in a similar manner except that “Expression” is selected as the Project Type in Field 510. The status of a project may be viewed at any time. A study is typically considered to be completed when the desired scientific objective has been accomplished.

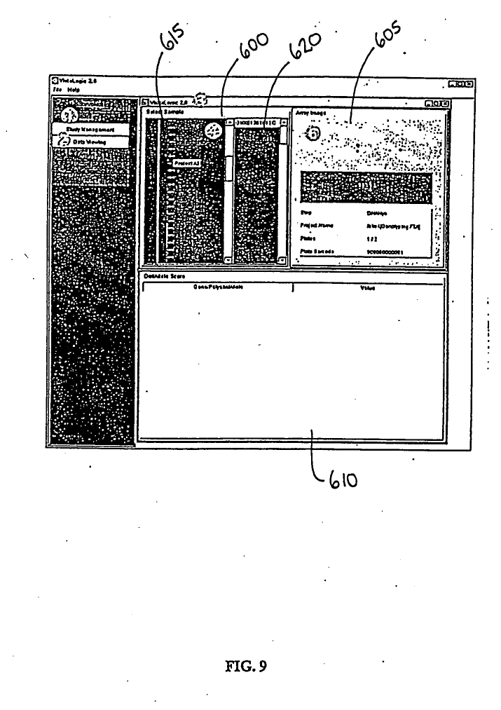

Project data may be viewed by the Client using the System Software as indicated in FIG. 9. The Data Viewing Window in FIG. 9 is divided into three main sections: (1) the Client/Study/Project Tree and Sample List in Section 600; (2) an Array Image Sample Information area in Section 605 designated for displaying detailed information about selected samples; and, (3) a Score Area in Section 610 for displaying allele scores for genotyping or expression scores for gene expression. The Client selects a Study and a Project in Tree Area 615 of Section 600. After a project is selected, the System Software retrieves the requested information from the server (or servers) and displays the sample set in Sample List Area 620 of Section 600. An array image will be shown in Section 605, and the scores will be displayed in Section 610. If a particular sample is selected, then detailed sample information may be shown along with the score data.

Analysis

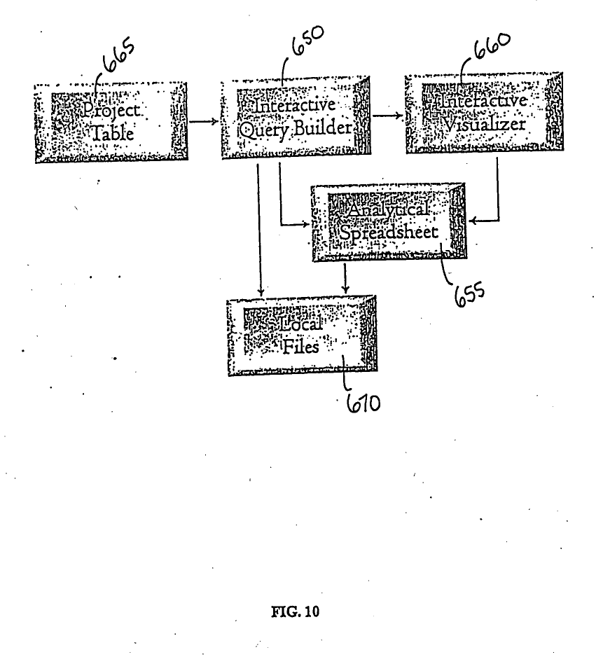

The System Software preferably provides analysis tools by which project data may be analyzed. Each table of information containing data generated within a project is analyzed individually, with each row in a project table corresponding to an individual sample. The columns may contain experimental data such as expression levels or allele scores, clinical or biological information about the samples, or sample identifiers such as the sample barcode identifier. Tools may be provided to analyze the data in the project table. Referring to FIG. 10, such tools may include: (1) Interactive Query Builder 650 allowing the Client to interactively build queries for selecting subsets of data for analysis; (2) Analytical Spreadsheet 655 allowing the Client to manually select subsets of data and to apply data analysis algorithms; and, (3) Interactive Visualizer 660 allowing the Client to interactively visualize selected data and the results of data analysis. Data from Project Table 665 may be transported sequentially from one tool to another or exported as Local File 670 as shown in FIG. 10.

Interactive Query Builder 650 preferably allows users to select rows (corresponding to samples) from Project Table 665 via several search techniques. For example, a fast search might allow the Client to select columns of data that will be used to formulate the search criterion. These selected columns could then be uploaded into Analytical Spreadsheet 655. A more detailed searching method could be provided in which a detailed query using Boolean operations could be created and stored for later reuse.

Analytical Spreadsheet 655 allows manual selection of subsets of data from Project Table 665. The selected subsets of data may then be used as input to analytical algorithms and visualization tools, or may be exported in the desired format for analysis using outside programs. Preferably, data may be selected for use or export by rectangular region, as well as by columns or by rows. Further, analytical algorithms may be generated directly from the spreadsheet by selecting the desired algorithm and the required input variables. For example, genotyping and outcome information could be analyzed using two-variable (odds ratio) and three-variable (Mantel-Haenszel) methods. As another example, genotyping and outcome information could be analyzed using a supervised learning algorithm that discovers predictive models (probabilistic rules) for predicting clinical outcomes from the genotyping information. Data from the analytical spreadsheet preferably may be exported into a local file using a variety of standard formats.

Data from Analytical Spreadsheet 655 may be transferred to Interactive Visualizer 660 for visual display as, for example, a scatter plot or a curve plot. A scatter plot may be provided for visualization of three selected columns: two numeric columns and a third column containing a small number (typically up to 16) of distinct values. Each dot in the scatter plot would then correspond to a row in the spreadsheet (ie., to an individual sample). The color of the dot could then indicate the value in the third column. For example, a scatter plot could be used to visualize a gene expression project in which a number of toxic and non-toxic compounds are applied to a particular cell line. Clustering of dots would then indicate distinct expression profiles. By selecting toxicity as the third column, one could then determine if the known toxicity of the administered compounds correlates with the clustering, Le., with the expression profiles.

A curve plot may be used for simultaneous visualization of multiple curves. The horizontal axis would typically be chosen to represent time, dosage, or some other numeric value. Multiple gene expression levels or other numeric values may be selected for multi-color display along the vertical axis. For example, a curve plot could be used to visualize a gene expression project in which the gene expression of a number of genes is simultaneously measured in a cell line at multiple time points after treatment with a particular compound. Time-dependent variation in gene expression levels of multiple genes may then be simultaneously visualized by displaying time on the horizontal axis and the measured gene expression levels for genes of interest along the vertical axis. As another example, a curve plot may be used for a gene expression project in which the gene expression of a number of genes is simultaneously measured in a cell line in response to treatment with different dosages of a particular compound. Dose-dependent variation in gene expression levels of multiple genes may be simultaneously visualized by displaying dosage along the horizontal axis and the measured gene expression levels for the genes of interest along the vertical axis.

Production

As described above, a project is a unit of experimental data collection, defined by a set of samples and a multiplex assay that is performed on the set of samples. Samples used in a project initially may be present in individual tubes or arrayed in microtiter plates. If the samples are not arrayed, the first step in a project involves the plating (arraying) of samples and the normalization of sample concentration. The subsequent processing of samples may then be tracked on a plate-by-plate basis. Plates (containing samples at different processing stages) may be imported from one project into the other. Samples may be tracked on a plate-by-plate basis even during imaging, dot-scoring, allele-scoring, and expression-scoring steps—well after the physical plates have ceased to exist in the process of converting physical objects into information.

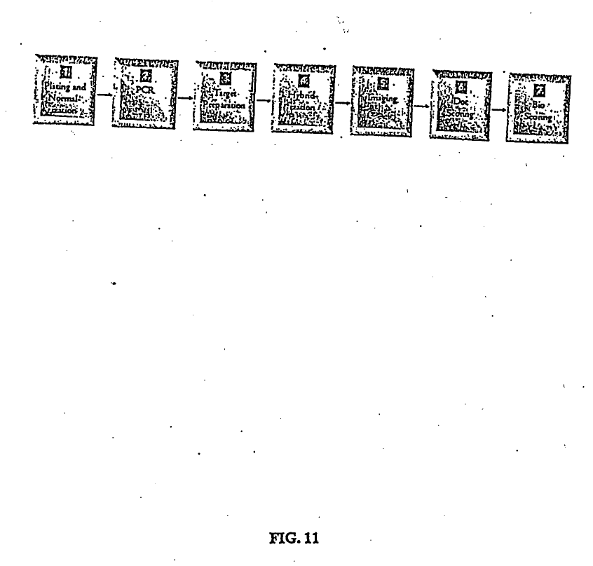

Each project typically consists of a number of processing steps (typically about 7) depending on the type of project (genotyping, gene expression, or proteomics). A typical genotyping project is shown in FIG. 11. A Production Manager will typically create work orders for each individual step and give the work orders to Workstation Operators. A Workstation Operator receiving a work order will typically enter the work order barcode into the workstation, enter the plate barcode, and initiate the process. The workstation communicates over a network with the database, receives details regarding the operation, performs the operation, and reports the successful completion.

Upon completion of an operation, the Production Manager will log the completion of the work orders. A work order may contain multiple sample plates. Each such plate may be in one of three states: In Process, Completed, or Failed. Each state is preferably represented by a different color code.

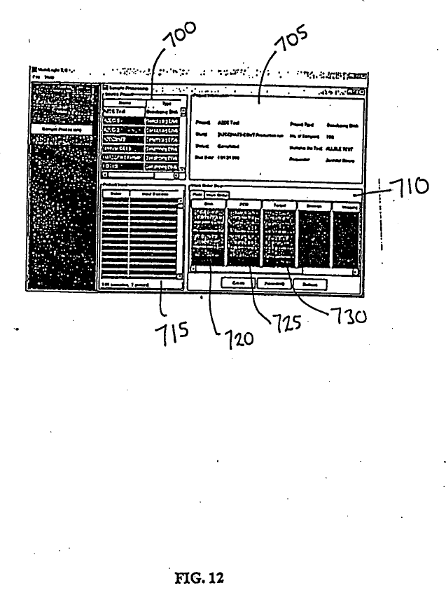

The System Software may be used to issue a work order via a Production Menu as shown in FIG. 12. Source Project Window 700 displays initiated projects that are not yet completed. Detailed information about each project may be displayed in Project Information Window 705 and Work Order Step Window 710. When applicable, individual samples may be listed in Project Input Window 715. For example, a project may contain completed plates in its first three steps: DNA plating and normalization, sample PCR, and target preparation. Since the DNA plating and normalization step is part of the project, the project was initiated with individual samples, which would be shown in Project Input Window 715. If the project were initiated with PCR plates instead, then DNA column 720 and PCR column 725 in Work Order Step Window 710 maybe shaded. Since Target Plate Column 730 is present in Work Order Step Window 710, the work order for hybridization is ready to be issued.

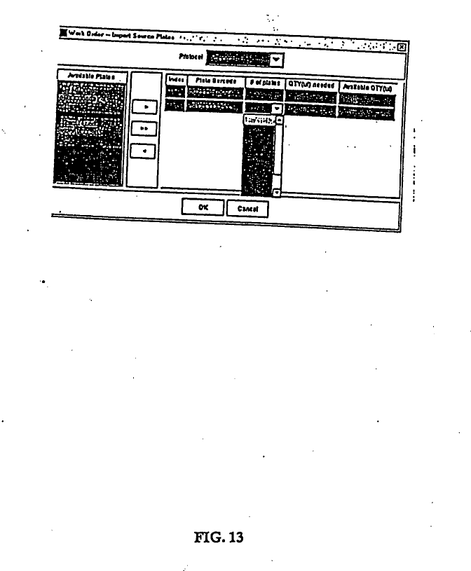

The Hybridization Step begins with the importation of source plates into a work order, as shown in FIG. 13, which displays a dialog box suitable for use with processing steps where physical plates are imported and created in the course of operation (e.g. PCR and target preparation). Different dialog boxes may be used for steps that do not use imported plates (e.g., plating and normalization) and steps where the output plate is not a physical plate containing samples but plate-related information (e.g., hybridization, imaging, dot scoring, and bio scoring).

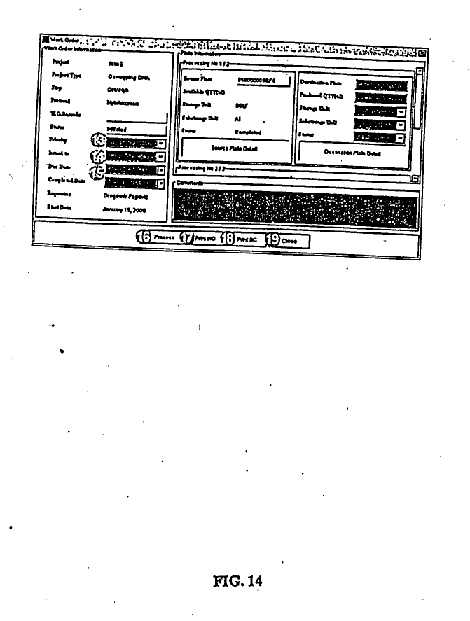

After source plates are imported, a Work Order Window with plate information may be presented as shown in FIG. 14. This window may be used to select a Work Station Operator, to assign a due date, and to assign a priority. It also may be used to print barcodes or to print the work order itself. A completion date may be entered to log the completion of a work order.

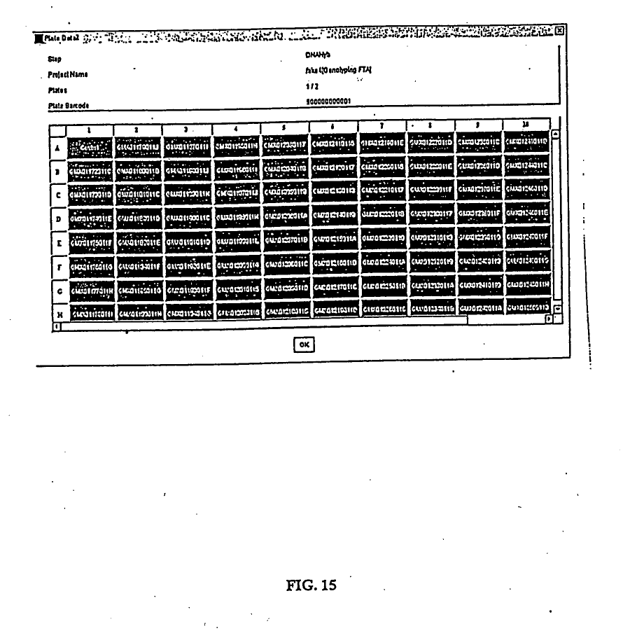

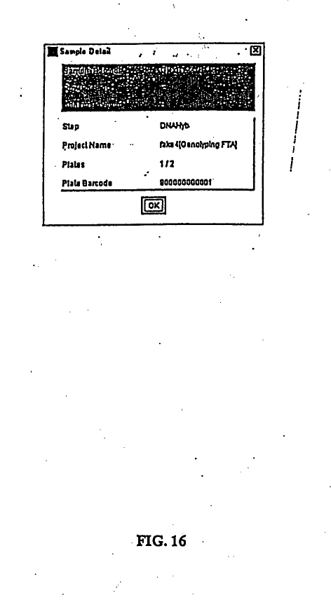

The System Software may be used to view the status of work orders, plates, and samples using the Production Menu of FIG. 12. Selecting a particular plate in FIG. 12 displays a Plate Detail Window as shown in FIG. 15. Selecting a particular sample in FIG. 12 displays a Sample Detail Window as shown in FIG. 16.

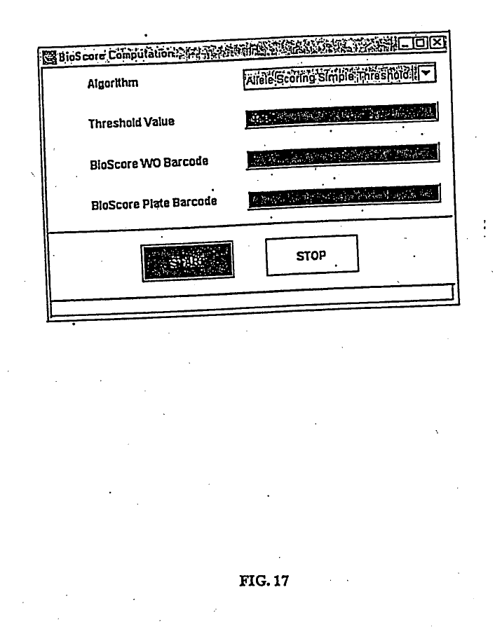

In the bio-scoring step, the DotScores are converted into biologically meaningful quantities (BioScores). A BioScore in a genotyping project is a genotype value (i.e., an allele score), whereas in a gene expression project it indicates a gene expression level. A BioScore computation may be initiated using the dialog in FIG. 17. A project is completed when a bio-scoring plate is completed. At that point, the data from the project is ready for viewing and analysis.

### Description of the Process Flow

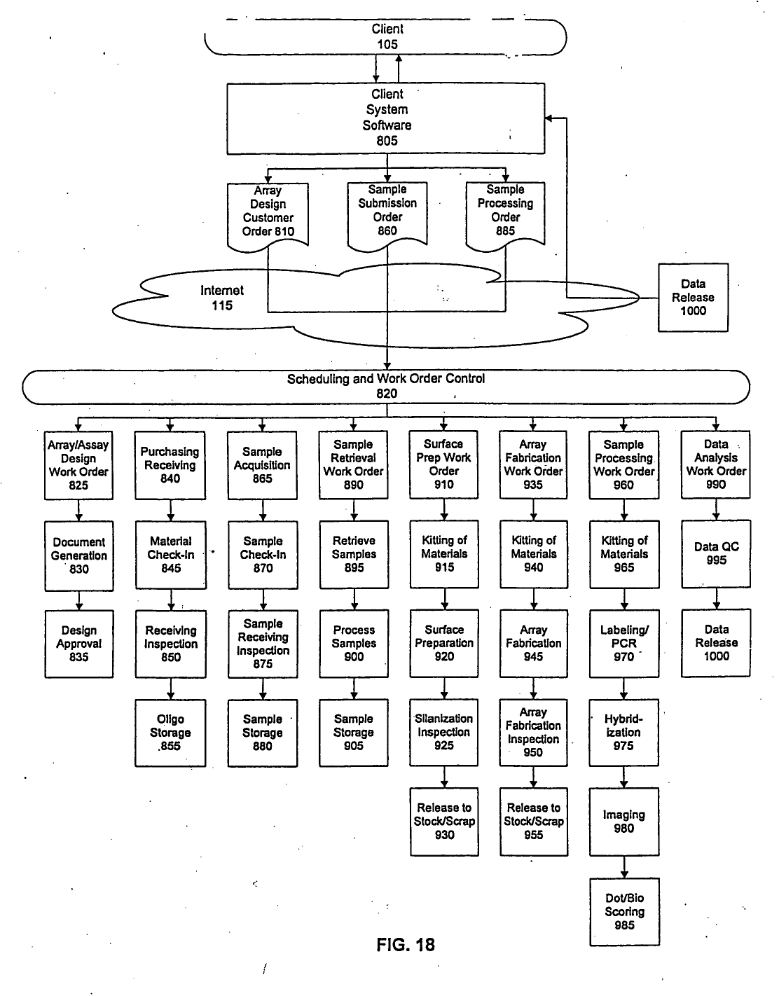

Referring to FIG. 18, a study or project is initiated when Client 105 uses System Software 805 over Internet 115 and submits Array Design Client Order 810. Scheduling and Work Order Control 820 generates Array/Assay Design Work Order 825 that results in Document Generation 830 and Design Approval 835. Array/Assay Design Work Order 840 is then transferred to Purchasing/Receiving 840, which handles Material Check-In 845, Receiving and Inspection 850, and Oligo Storage 855. Client 105 again uses System Software 805 over Internet 115 and submits Sample Submission Order 860. The resulting Sample Acquisition stage 865 involves Sample Check-In 870, Sample Receiving and Inspection 875, and Sample Storage 880. Client 105 uses System Software 805 over Internet 115 and submits Sample Processing Order 885. Scheduling and Work Order Control 820 generates Sample Retrieval Work Order 890 resulting in Sample Retrieval 895, Sample Processing 900, and Sample Storage 905. Next, Surface Preparation Work Order 910 is generated, resulting in the Material Kitting 915, Surface Preparation 920, Silanization Inspection 925, and Release to Stock/Scrap 930. Array Fabrication Work Order 935 results in Material Kitting 940, Array Fabrication 945, Array Fabrication Inspection 950, and Release to Stock/Scrap 955. Sample Processing Work Order 960 results in Material Kitting 965, Labeling/PCR 970, Hybridization 975, Imaging 980, and Dot/Bio Scoring 985. The results from Dot/Bio Scoring 985 are transferred to Data Analysis Work Order 990 resulting in Data QC 995 and Data Release 1000 to System Software 805 over Internet 115 and back to Client 105.

### Conclusion

Although the processes described herein have used the Internet for the submission of the work order, the selection of the biological samples, and the delivery of data, the present invention is not limited to the use of the Internet. For example, the samples and the work order may be submitted by mail or the results may be provided in the form of a computer file on a compact disc. The format of the computer file may be chosen so that the Client can import the data into its own analysis software. For example, if the Client analyzes the data using an electronic spreadsheet program, then the data may be supplied over the Internet or on a compact disc in the form of an electronic spreadsheet.

The present invention, therefore, is well adapted to carry out the objects and obtain the ends and advantages mentioned above, as well as others inherent herein. All presently preferred embodiments of the invention have been given for the purposes of disclosure. Where in the foregoing description reference has been made to elements having known equivalents, then such equivalents are included as if they were individually set forth. Although the invention has been described by way of example and with reference to particular embodiments, it is not intended that this invention be limited to those particular examples and embodiments. It is to be understood that numerous modifications and/or improvements in detail of construction may be made that will readily suggest themselves to those skilled in the art and that are encompassed within the spirit of the invention and the scope of the appended claims.

I claim:

1. A method of requesting genomics services from a service provider and providing genomics services to a client comprising:
under control of said client,
providing one or more biological samples;
identifying one or more genome sequences;
under control of said service provider,
obtaining said samples and said genome sequences;
providing one or more microarrays wherein each of said microarrays contains at least one of said genome sequences;
applying at least one of said biological samples to at least one of said microarrays; and,
under control of said client,
receiving data representative of said applying step over the Internet.

2. The method of claim I wherein said genome sequence comprises a single nucleotide polymorphism.

3. The method of claim 1 wherein said step of providing one or more biological samples comprises:
under control of said service provider;
providing a repository of biological samples;
providing a catalog of said repository;
under control of said client;
accessing said catalog; and,
selecting said biological samples from said catalog.

4. The method of claim 3, wherein said steps of providing a catalog and accessing said catalog occur over an Internet connection between said client and said service provider.

5. The method of claim 4 wherein said Internet connection is a secure Internet connection.

6. The method of claim 1 wherein said one or more biological samples comprises tissue samples.

7. The method of claim 1 wherein said one or more biological samples comprises DNA samples.

8. The method of claim 1 further comprising the step of analyzing said data representative of said applying step.

9. The method of claim 8 wherein said analyzing step occurs under control of said client.

10. The method of claim 8 wherein said analyzing step occurs under control of said service provider.

11. The method of claim 1 wherein said data representative of said applying step comprises genotype data.

12. The method of claim 1 wherein said step of providing one or more biological samples comprises transporting said samples from said client to said service provider.

13. The method of claim 12 wherein said data representative of said applying step comprises gene expression data.

14. The method of claim 12 wherein said one or more biological samples comprises total RNA samples.

15. The method of claim 12 wherein said one or more biological samples comprises poly-A RNA samples.

16. A method of requesting proteomics services from a service provider and subsequently providing proteomics services to a client comprising:
under control of said client,
providing one or more biological samples;
identifying one or more antigens;
under control of said service provider,
obtaining said samples and identified antigens;
providing one or more microarrays wherein each of said microarrays contains probes to detect at least one of said identified antigens;
applying at least one of said biological samples to said microarrays; and,
under control of said client,
receiving data representative of said applying step over the Internet.

17. The method of claim 16 wherein said step of providing one or more biological samples comprises:
under control of said service provider;
providing a biological sample repository;
providing a catalog of said biological repository;
under control of said client;
accessing said catalog; and,
selecting said biological samples from said catalog.

18. The method of claim 17, wherein said steps of providing a catalog and accessing said catalog occur over an Internet connection between said client and said service provider.

19. The method of claim 18 wherein said Internet connection is a secure Internet connection.

20. The method of claim 1 or claim 16 further comprising applying a unique identifier to each of said one or more biological samples.

21. The method of claim 20 wherein said applying step is performed under control of said client.

22. The method of claim 20 wherein said applying step is performed under control of said service provider.

23. The method of claim 20 further comprising tracking said one or more biological samples using said unique identifier.

24. The method of claim 23 wherein said tracking step is performed under control of said client.

25. The method of claim 23 wherein said tracking step is performed under control of said service provider.

26. A process for remotely selecting samples from a biological repository comprising:
under control of a service provider;
providing a database of said samples in said biological repository;
providing a network connection to said database accessible by a client;
under control of a client;
accessing said database over said network; and,
selecting a subset of said samples from said biological repository.

27. The process of claim 26 wherein said network is the Internet.

28. The process of claim 26 wherein said database includes clinical records corresponding to at least a portion of said samples.

29. The process of claim 26 wherein said database includes phenotype information corresponding to at least a portion of said samples.

30. The process of claim 26 wherein said database includes follow-on medical history information corresponding to at least a portion of said samples.

31. A process for remotely conducting a genomics experiment comprising:
under control of a service provider;
providing a database of biological samples in a biological repository;
providing a network connection to said database accessible by a client;
under control of a client;
accessing said database over said network;
selecting a subset of said samples from said biological repository;
identifying a set of genomic sequences;
under control of said service provider;
determining if said genomic sequences are present in said samples; and,
informing said client of results of said determining step.

32. The process of claim 31 further comprising under control of said service provider:
modifying said set of genomic sequences subsequent to said informing step.

33. The process of claim 31 further comprising identifying said- samples with unique identifiers.

34. The process of claim 33 further comprising
under control of said client:
selecting a sample from said subset of samples;
determining a unique identifier corresponding to said selected sample;
requesting status information regarding said identifier;
under control of said service provider:
determining status of said identifier; and,
informing said client of said status.

35. A method of providing genomics services to a client comprising:
receiving one or more biological samples from said client;
receiving one or more genome sequences from said client;
providing one or more microarrays wherein each of said microarrays contains at least one of said genome sequences;
applying at least one of said biological samples to at least one of said microarrays; and,
transmitting data representative of said applying step to said client over the Internet.

36. A method of providing genomics services to a client comprising:
receiving one or more biological samples from said client over the Internet;
receiving one or more genome sequences from said client;
providing one or more microarrays wherein each of said microarrays contains at least one of said genome sequences;
applying at least one of said biological samples to at least one of said microarrays; and,
transmitting data representative of said applying step to said client.

37. A method of providing genomics services to a client comprising:
receiving one or more biological samples from said client;
receiving one or more genome sequences from said client over the Internet;
providing one or more microarrays wherein each of said microarrays contains at least one of said genome sequences;
applying at least one of said biological samples to at least one of said microarrays; and,
transmitting data representative of said applying step to said client.

38. A method for providing experimental biological services to a client comprising:
receiving a work order from said client comprising a biological sample portion listing one or more biological samples and an assay portion listing one or more experiments based on said biological samples;
performing said one or more experiments on said one or more biological samples; and,
transmitting data representative of said performing step to said client.

39. The method of claim 38 wherein said biological sample portion of said work order comprises one or more biological samples submitted by said client.

40. The method of claim 38 wherein said biological sample portion of said work order comprises one or more pointers to records in a biological sample database.

41. The method of claim 38 wherein said assay portion of said work order comprises an experimental protocol and a specification of one or more probes.

42. The method of claim 41 wherein said performing step further comprises:
providing one or more microarrays onto which said one or more probes is deposited; and,
interrogating said one or more biological samples with said one or more microarrays.

43. The method of claim 42 wherein said one or more probes are immobilized to a substrate.

44. The method of claim 43 wherein said one or more probes are immobilized on a flat surface.

45. The method of claim 43 wherein said one or more probes are immobilized in a three-dimensional polymer.

46. The method of claim 43 wherein said one or more probes are immobilized on bead surfaces.

47. The method of claim 42 wherein said one or more probes are in solution.

48. The method of claim 38 wherein said receiving step further comprises receiving said biological sample portion of said work order from a remote location.

49. The method of claim 38 wherein said receiving step further comprises receiving said assay portion of said work order from a remote location.

50. The method of claim 38 wherein said transmitting step further comprises transmitting said data to a remote location.

51. The method of claim 38 wherein said receiving step further comprises receiving said biological sample portion of said work order over the Internet.

52. The method of claim 38 wherein said receiving step further comprises receiving said assay portion of said work order over the Internet.

53. The method of claim 38 wherein said transmitting step further comprises transmitting said data over the Internet.

54. The method of claim 38 wherein said one or more biological samples comprises tissue samples.

55. The method of claim 38 wherein said one or more biological samples comprises DNA samples.

56. The method of claim 38 wherein said one or more biological samples comprises total RNA samples.

57. The method of claim 38 wherein said one or more biological samples comprises poly-A RNA samples.

58. The method of claim 38 wherein said assay portion of said work order comprises a list of genes.

59. The method of claim 38 wherein said assay portion of said work order comprises a list of single nucleotide polymorphisms.

60. The method of claim 38 wherein said assay portion of said work order comprises a list of proteins.
